(19) United States
(12) Patent Application Publication
(10) Pub. No.: US 2007/0048729 A1
(43) Pub. Date: Mar. 1, 2007

(54) Method of creating a collection of biological specimens, and collection of specimens

(76) Inventor: Hartmut Juhl, Hamburg (DE)

Correspondence Address: Raymond J Lillie;Carella Byrne Bain Gilfillan Cehhi Stewart & Olste, 5 Becker Farm, Roseland, NJ, 07068, US

(21) Appl. No.: 10/539,338

(22) PCT Filed: Dec. 19, 2003
(86) PCT No.: PCT/DE03/04217

§ 371 Date: Jun. 15, 2005

(30) Foreign Application Priority Data
Dec. 23, 2002 (DE) 102 61 529.2

(51) Int. Cl. C12Q 1/00; C12N 5/08

(52) U.S. Cl. 435/4; 435/366

(57) ABSTRACT

The invention relates to a method of creating a collection of isolated biological specimens, wherein each isolated biological specimen is preserved within a defined length of time following isolation of the specimen from its natural environment and is subsequently stored, and wherein the defined length of time between isolation and preservation of the various specimens has a defined maximum deviation. The invention also relates to a collection of biological specimens.

The invention relates to a method of creating a collection of biological specimens and to a collection of isolated biological specimens.

It is known from numerous publications, for example Alon et al., Proc. Natl. Acad. Sci. USA Vol. 96 (1999) 6745-6750; Zou et al., Oncogene 21 (2002) 4855-4862; Nottermann et al., Cancer Research 61 (2001) 3124-3130, Sorlie et al., PNAS 98 (2001) 10869-10874, that isolated biological tissue specimens can be shock-frozen in liquid nitrogen and stored at approximately −170° C. or −80° C.

One drawback of these known methods is that the biological tissue specimens are not isolated, prepared, preserved, and stored under standardized conditions. On account of the lack of standardization, experimental results which have been obtained in experiments on various isolated biological specimens are not sufficiently comparable with one another.

The time elapsing between isolation of a biological specimen from its natural environment and preservation or freezing of the biological specimen has a significant influence on the biochemical state or the condition of the isolated biological specimen. A biological specimen removed from a human, for example tumorous material, changes on account of the lack of supply of nutrients by the blood circulation. For example, a breakdown of nucleic acids, in particular ribonucleic acids, and of proteins occurs. Modification, for example phosphorylation and/or dephosphorylation of cellular constituents, in particular proteins, can also occur.

That is to say, with an increasing length of time following isolation of the biological specimen, the isolated biological specimen is no longer in the same bio-chemical or physiological state it was prior to its removal from its natural environment.

When carrying out experimental in vitro investigations on isolated biological specimens, it is essential for the isolated biological specimen to reflect the in vivo conditions to make it possible to get results which allow a statement about the biochemical, physiological and/or molecular biological in vivo conditions.

Such an isolated biological specimen would, for example, be a valuable investigation material for developing active pharmaceutical ingredients and drugs in the field of cancerous or metabolic diseases.

In addition, such a high-grade biological specimen would also be very suitable for investigating the molecular biological and/or pathobiochemical processes in pathological biological specimens in comparison with non-pathological biological specimens, in order to obtain knowledge about the molecular causes of diseases, for example cancerous or metabolic diseases.

In order to be able to assess the relevance of experimental findings, they must be confirmed statistically. In this regard, an appropriate number of experimental investigations on isolated biological specimens of various origin must be carried out. A prerequisite in this case is that the various isolated biological specimens be prepared, preserved, and stored under standardized conditions after isolation.

There is accordingly a need for a method of creating a collection of biological specimens.

In addition, there is a need for a collection of biological specimens which reliably reflects the biochemical state in its natural environment.

The object on which the invention is based is achieved by a method of creating a collection of biological specimens, wherein isolated biological specimens are preserved within a defined length of time following isolation of the specimen from its natural environment and are subsequently stored and wherein the defined length of time between isolation and preservation of various specimens has a defined maximum deviation.

Preferred refinements of the method according to the invention are indicated in subclaims 2 through 15.

The object is furthermore achieved by a specimen collection which contains biological specimens isolated and prepared as set forth in the method as claimed in any one of claims 1 through 15.

The process of taking or isolating the biological specimen from its natural environment, for example by surgical intervention in humans, is not subject matter of the invention. The method of the invention for creating a specimen collection of isolated biological specimens follows immediately upon taking a specimen and can be carried out by laboratory personnel without medical supervision.

According to a preferred embodiment, the condition of the biological specimen following isolation from its natural environment and before preservation is recorded and documented.

The condition of the isolated biological specimen can be recorded, for example, by means of photographic documentation. In addition, a medical or scientific appraisal and assessment of the state of the isolated biological specimen and documentation of the assessment can take place. Recording of the condition of the biological specimen immediately after isolation from its natural environment allows a more comprehensive assessment and evaluation of investigations and/or experiments carried out on the isolated biological specimen at a later point in time.

Preferably, the biological specimen has a defined volume. In this case, a volume of approximately 0.5 cm³ to approximately 1 cm³ has proven very suitable. It is preferred here to obtain a number of biological specimens which have approximately the same volume, for example approximately 0.5 cm³ and/or approximately 1 cm³. Of course, the biological specimen can also occupy smaller volumes, for example 1 mm³ or 3 mm³, or alternatively larger volumes, for example 2 cm³ or 4 cm³.

Immediately after isolation from its natural environment and recording of its condition, for example by means of digital photographic documentation, the biological specimen can be trimmed to the desired specimen volume using a scalpel. Subsequently, the specimen volumes can be transferred to suitable specimen tubes, for example cryotubes. The cryotubes can then be stored in liquid nitrogen.

Alternatively, the isolated biological specimens can be embedded in paraffin. Optionally, before embedding the specimen in paraffin, dehydration thereof under standardized conditions can take place. It is possible, for example, to prepare from the embedded specimens tissue sections for microscopic examination.

According to a further embodiment of the invention, the defined maximum deviation from the defined period of time is not more than approximately 10%, preferably not more than approximately 5%, based on the defined period of time.

It has been found that keeping to a defined period of time, measured from the moment of taking the biological specimen up to preservation and/or storage of the isolated biological specimen, greatly improves the comparability of the condition of the isolated biological specimens.

By keeping to standardized conditions during down-stream processing of the isolated biological specimens, in particular the period of time between taking the specimen and preserving and/or storing the same, the biological specimens thus collected show very good comparability.

Accordingly, when comparing the biochemical and/or physiological state of the isolated biological specimens of, say, healthy or nonpathological tissue specimens with that of pathological tissue specimens, the differences can be attributed to the respective disease or degeneration. That is to say, with standardized processing of the isolated biological specimens, any differences that may be found between various biological specimens will be attributable not to the respective processing method or to a different period of time of processing, but to an indication of the molecular causes of the respective disease or degeneration.

According to the invention, it is preferred that the defined period of time be less than approximately 25 minutes, and preferably less than approximately 15 minutes. More preferably, the defined period of time is approximately 12 minutes. Most preferably, the defined period of time is approximately 10 minutes. Of course, the defined period of time can be shorter, for example 5 or 8 minutes, if desired.

With regard to the fact that the isolation of human biological specimens has to be carried out in an operating theatre and processing of the isolated biological specimens normally takes place outside the operating theatre, it is virtually impossible to reduce the period of time, as measured from isolation of the biological specimen until preservation of the isolated biological specimen, to less than five minutes. A defined period of time of approximately 10 minutes has been found to be very suitable.

According to a further preferred development, preservation of the isolated biological specimen is effected by cryopreservation or by chemical preservation.

“Preservation” is understood within the scope of the invention as meaning that the biochemical or physiological state of the isolated biological specimen becomes fixed.

Cryopreservation is preferably carried out by immersing the isolated biological specimen in a cryogenic medium and freezing the biological specimen over a period of time of, preferably, a few seconds. The cryogenic medium used is preferably liquid nitrogen. In this way, preservation and storage of the isolated biological specimen coincide.

According to another variant of the method of the invention, preservation is carried out using chemical crosslinking agents. The crosslinking agents preferably used have reactive groups.

“Reactive groups” are understood within the scope of the invention as meaning chemical functionalities which react chemically with the isolated biological specimen. In this case, reactive groups of the crosslinking agent can react with reactive groups on the isolated biological specimen. Preferably, the crosslinking agent used in the invention contains aldehyde and/or epoxide groups, which preferably react with amino groups on the isolated biological specimen.

Said crosslinking agents are preferably selected from the group consisting of formaldehyde, polyaldehydes, preferably dialdehydes, polyepoxide compounds, preferably diepoxide and/or triepoxide compounds, and mixtures thereof.

Preferably, the dialdehyde used is glutaraldehyde. Paraformaldehyde can of course be used as an alternative to formaldehyde.

The triepoxide compounds used can be, for example, polyalkylene glycol diglycidyl ethers, preferably polyethylene glycol diglycidyl ether, or alkanediol glycidyl ethers, for example 1,6-hexanediol glycidyl ether and/or 1,4-butanediol glycidyl ether.

The polyglycidyl compounds used can be, for example, polyalcohol polyglycidyl ethers, for example sorbitol polyglycidyl ethers, glycerol polyglycidyl ethers, pentaerythritol polyglycidyl ethers, saccharide polyglycidyl ethers, and mixtures thereof.

The isolated biological specimen can be preserved only by cryotreatment or only by chemical preservation. Of course, the isolated biological specimen can be initially treated with a chemical cross-linking agent or preservative and subsequently subjected to a cryotreatment. Alternatively, however, the isolated biological specimen can be initially subjected to a cryotreatment and stored under liquid nitrogen or in a freezer, for example at −80° C., and at a later point in time subjected to chemical preservation by treatment with a chemical cross-linking agent or preservative.

Preferably, the isolated biological specimen is human tissue. Theoretically, the biological specimen used in the method of the invention can be of any biological material for which it is desired to create a collection of isolated biological specimens.

Tumor-free tissue, tumor tissue and/or fatty tissue are particularly suitable for the method of the invention. In addition, it is preferred that the tumor tissue be central or peripheral tumor tissue. The tumor tissue used can be, for example, tissue of colon carcinoma, rectal carcinoma, pancreatic carcinoma, mammary carcinoma, prostatic carcinoma, bronchiolar carcinoma, gastric carcinoma, or cervical carcinoma.

Comparison of the biochemical and/or physiological state of tumor-free tissue, central or peripheral tumor tissue taken from the specimen collection of the invention or of isolated biological specimens prepared by the method of the invention can demonstrate the molecular differences, for example, of gene activities, expression patterns, expression profiles, activated proteins, in particular cellular tumor factors, enzymes, etc., in experimental investigations.

The different DNA, RNA and/or protein activities in the isolated biological proteinaceous specimens can be investigated, for example, in microarray analyses, for example on “biochips” (DNA arrays or protein arrays).

It is possible, from this comparison of biological specimens prepared under standardized conditions, to determine possible sites of action for active compounds and/or drugs in the treatment of cancer or metabolic diseases. In particular, a statistical validation of the experimental results is possible using a plurality of isolated biological specimens processed by the method of the invention.

According to an advantageous development, datasets are assigned to the isolated biological specimens.

The datasets contain, in particular, information on the isolated specimens. As a rule, the isolated biological specimen is divided into a number of pieces prior to, or following, preservation and the pieces are stored separately from one another. Some of the specimens can then be analyzed using molecular biological methods, for example at the protein level and/or mRNA level. These data allow a statement on the state of activation or inactivation of genes, mRNAs and/or proteins. Using these data, an activation profile or expression profile of the isolated biological specimen can thus be created.

The assignment of the datasets to the isolated biological specimens can be effected, for example, by way of identification numbers of the respective isolated biological specimens in a computer-managed database.

Preferably, the datasets comprise further information on the case history, medication, anesthesia, course of the operation, clinical parameters, and/or aftercare data.

The datasets preferably additionally contain information on clinicochemical diagnoses of blood, stool, urine, sputum, cerebrospinal fluid samples, etc. obtained prior to and/or following isolation of the respective biological specimen from the patient. Thus the datasets can contain information on the blood group, blood picture, clotting values, tumor markers, liver values, kidney values, serum electrolyte values, etc.

In addition, the datasets can contain information on drugs administered to the patient prior to and/or following the isolation of biological specimens.

Likewise, the datasets can contain information regarding the case history, for example eating and living habits such as appetite, aversions, allergies, previous diseases, pediatric diseases, infectious diseases, tropical diseases, earlier cancerous diseases, sleeping habits, stool excretion habits, urine discharge habits, consumption of alcohol, nicotine, and/or drugs, complaints and health conditions, symptoms, drug dosage, and intolerance of pharmaceuticals, etc.

In addition, the datasets can hold information on the time line involved in removing the biological specimen from its natural environment. Preferably, the commencement of the time line is the moment of separation or excision of the tissue from a human. When isolating colon tissue specimens, the moment of parting the proximal and distal ends of the biological specimen represents the starting point for time measurement.

In particular, further information regarding the isolated biological specimen can be documented, for example particulars regarding the size of the material from which tissue was isolated, for example the tumor size.

The isolated biological specimens can then be preserved by cryotreatment, for example in liquid nitrogen, as explained above, and stored under liquid nitrogen or in a freezer, for example at approximately −80° C.

Alternatively, the isolated biological specimens can be first of all chemically preserved or fixed and, if desired, subsequently subjected to cryotreatment, for example by treatment with liquid nitrogen, and finally stored until further use, for example, under liquid nitrogen or in a freezer, for example at approximately −80° C.

The method of the invention allows for the creation of a collection of isolated biological specimens which have been processed under standardized conditions, it being possible to assign a multiplicity of clinically relevant data to each isolated biological specimen. The combination of standardized specimens and clinically relevant data is extremely valuable for research on active ingredients or pharmaceuticals.

Preferably, the specimen collection according to the invention holds more than 100, preferably more than 500, and more preferably more than 1000, isolated biological specimens.

### EXAMPLE 1

### Creating a Collection of Isolated Colon Tissue

The method of the invention for creating a collection of isolated biological specimens is illustrated below with reference to isolated colon tumor tissue. However, the method of the invention is not restricted to colon tumor tissue or colon tissue and can of course be implemented for other body tissue, for example bronchial tissue, mammary tissue, etc.

For the purpose of making datasets assigned to the isolated biological specimens, not only the case history data and further clinicochemical parameters of the patient, for example analyses of blood, urine, cerebrospinal fluid, sputum, etc., but also the time line up to and including the moment of taking the biological specimen was documented. Accordingly, specimens obtained under standardized conditions by the method of the invention are prepared for the creation of a specimen collection. Moreover, a multiplicity of clinically relevant information relating to the patient from whom the specimen was taken and to the production of the specimen itself and to analytical data of the isolated specimen is preferably assigned to the isolated biological specimen and stored in the form of a dataset.

Accordingly, on the one hand a multiplicity of biological specimens obtained within an extremely short time, for example 10 minutes, and under standardized conditions, and on the other hand specific information associated with the respective specimen, are available for, say, research on active ingredients or drugs.

Any differing results obtained during active ingredient research on different isolated tissue specimens, for example colon tissue specimens, can possibly be understood, explained, and/or interpreted against the background of the other available information. The specimen and data collection of the invention created using the method of the invention is an extremely valuable tool, in particular for active ingredient research in the pharmaceutical industry.

In Table 1 below, a minus sign is placed in front of the times prior to isolation of the specimen. The process of taking the specimen is not part of the method of the invention. Rather, the method of the invention begins immediately after isolation of the biological specimen has taken place. A plus sign is placed in front of the times following isolation in Table 1.

TABLE 1
Time line from the moment of taking a specimen of colon tissue up
to preservation thereof

| Time | | Action |
|---|---|---|
| −77 | min | Commencement of anesthesia of the patient |
| −61 | min | Blood collection |
| −45 | min | Commencement of operation |
| −38 | min | Urine collection |
| −14 | min | Tying off of the mesenterica inferior |
| −7 | min | Tying off of the lower arch |
| −5 | min | Tying off of the upper arch |
| −3 | min | Severing the distal end of the resectate (colon tissue) |
| 0 | min | after separation of the proximal end of the resectate |
| +1 | min | Resectate is excised along the course of the intestine using |
| | | scissors, the tumor preferably not being transected. |
| +1-5 | min | The resectate and tumor are preferably photographed with a |
| | | digital camera. A close-up of the tumor is made. |
| +5 | min | Specimens are taken from the resectate and tumor. The |
| | | specimens comprise, for example, healthy tissue, adipose |
| | | tissue, peripheral tumor tissue, and central tumor tissue. |
| | | The healthy tissue is removed from the resectate at a |
| | | distance of at least 5 cm from the tumor. The specimens |
| | | are divided up into pieces having a volume of preferably |
| | | approximately 0.5 cm³. The divided specimens are |
| | | transferred to specimen tubes. |
| +10 | min | Preservation of the specimens in the tubes: |
| | | by freezing in liquid nitrogen or |
| | | by addition of 10 ml of 3.5% strength formaldehyde |
| | | solution or |
| | | by addition of 10 ml of 5.5 wt % strength glutaraldehyde |
| | | solution. |

Preferably, groups each comprising a third of the specimens obtained by division of the isolated specimens are preserved by each of the above preservation methods and subsequently stored. In the case of preservation by means of formaldehyde or glutaraldehyde, the biological specimen is preferably allowed to stand at room temperature (eg 25° C.) for a defined period of time, for example for 10 hours or 24 hours. The addition of the preservative solution, ie formaldehyde solution, glutaraldehyde solution, or liquid nitrogen, marks the termination of the procedure of the invention.

### EXAMPLE 2

### Detection of the Change in Biological Specimens Over Time following Isolation thereof from their Natural Environment

As proof of the significance of the method of the invention, the protein composition of intestinal tissue samples was investigated, at defined time intervals following removal from the patient, by means of SELDI-MS (Surface-enhanced Laser Desorption Ionization Mass Spectrometry).

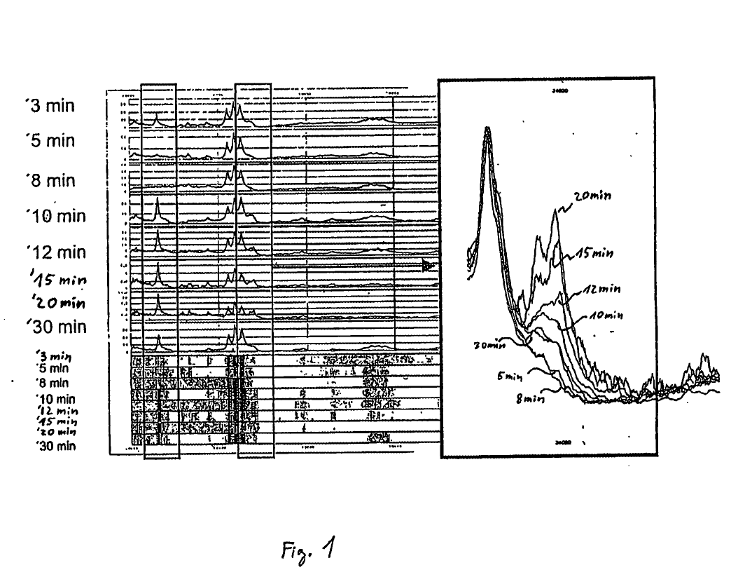

The result of SELDI-MS analyses of colon tissue samples is shown in FIG. 1. In this test, the specimen was frozen in liquid nitrogen 3 min, 5 min, 8 min, 10 min, 15 min, 20 min, and 30 min after it was taken from the patient. The samples were subsequently processed according to the manufacturers' instructions (CIPHERGEN, Gottingen, Germany) and analyzed by means of SELDI-MS.

The mass spectrum obtained in each case is shown in FIG. 1. An enlarged super-imposition of the mass spectra at various points in time following specimen-taking is taken from that part of the mass spectrum which is depicted in the right-hand frame and is shown in the box to the right of the mass spectra. The respective points in time at which freezing of the specimen in liquid nitrogen is effected are indicated on the superimposed curves depicted in said box showing the mass spectra obtained.

It is clearly seen that the protein composition in the isolated resectates, the isolated colon tissue samples, changes with time within minutes between resection and freezing. This change with time can be attributed, for example, to an over-regulation and/or underregulation of protein levels, for example as a result of oxygen deficiency (hypoxia).

These results confirm the great importance of the method of the invention, namely the standardization of processing of the isolated biological specimens when creating a collection of biological specimens.

1. A method of creating a collection of isolated biological specimens, wherein each isolated biological specimen is preserved within a defined length of time following isolation of the specimen from its natural environment and is subsequently stored, and wherein the defined length of time between isolation and preservation of the various specimens has a defined maximum deviation.

2. A method as defined in claim 1,
characterized in that
the condition of each biological specimen following isolation thereof from its natural environment and prior to preservation thereof is recorded and documented.

3. A method as defined in claim 1 or claim 2,
characterized in that
said biological specimen has a defined volume.

4. A method as defined in any one of claims 1 through 3,
characterized in that
said defined maximum deviation from said defined period of time is not more than approx. 10%, preferably not more than approx. 5%, based on said defined period of time.

5. A method as defined in any one of claims 1 through 4,
characterized in that
said defined period of time is shorter than approximately 25 minutes, preferably shorter than approximately 15 minutes.

6. A method as defined in any one of claims 1 through 5,
characterized in that
said defined period of time is approximately 12 minutes.

7. A method as defined in any one of claims 1 through 5,
characterized in that
said defined period of time is approximately 10 minutes.

8. A method as defined in any one of claims 1 through 7,
characterized in that
preservation is effected by cryopreservation or by chemical preservation.

9. A method as defined in claim 8,
characterized in that
said chemical preservation involves the use of a crosslinking agent having reactive groups.

10. A method as defined in claim 9,
characterized in that
said crosslinking agent is selected from the group consisting of formaldehyde, polyaldehydes, preferably dialdehydes, polyepoxide compounds, preferably diepoxide and/or triepoxide compounds, and mixtures thereof.

11. A method as defined in any one of claims 1 through 10,
characterized in that
said isolated biological specimen is human tissue.

12. A method as defined in claim 11,
characterized in that
said human tissue is tumor-free tissue, tumor tissue and/or adipose tissue.

13. A method as defined in claim 12,
characterized in that
said tumor tissue is central or peripheral tumor tissue.

14. A method as defined in any one of claims 1 through 13,
characterized in that
data sets are assigned to said specimens.

15. A method as defined in claim 14,
characterized in that
said data sets contain information on the case history, medication, anesthesia, course of the operation, clinical parameters, and/or after-care data.

16. A collection of biological specimens, containing isolated biological specimens which have been processed by the method defined in any one of claims 1 through 15.
